(19) United States
(12) Patent Application Publication
(10) Pub. No.: US 2024/0390071 A1
(43) Pub. Date: Nov. 28, 2024

(54) SYSTEMS AND METHODS FOR TARGET NODULE IDENTIFICATION

(71) Applicant: Intuitive Surgical Operations, Inc., Sunnyvale, CA (US)

(72) Inventors: Bai Wang, Palo Alto, CA (US); Shiyang Chen, Sunnyvale, CA (US); Sabrina A. Cismas, Saratoga, CA (US); Joy Janku, San Francisco, CA (US); Sida Li, San Jose, CA (US); Hui Zhang, San Jose, CA (US)

(21) Appl. No.: 18/696,292

(22) PCT Filed: Sep. 20, 2022
(86) PCT No.: PCT/US2022/076696

§ 371 Date: Mar. 27, 2024

Related U.S. Application Data
(60) Provisional application No. 63/249,096, filed on Sep. 28, 2021.

(51) Int. Cl. A61B 34/10; A61B 10/04; A61B 34/30; G06T 7/00; G06T 7/11; G16H 20/40

(52) U.S. Cl. CPC A61B 34/10; G06T 7/0012; G06T 7/11; G16H 20/40; A61B 10/04; A61B 2034/107; A61B 2034/303; G06T 2207/20104; G06T 2207/20156; G06T 2207/30064

(57) ABSTRACT

A system may comprise one or more processors and memory having computer readable instructions stored thereon. The computer readable instructions, when executed by the one or more processors, may cause the system to receive image data including a segmented candidate target nodule, receive a seed point, and determine if the segmented candidate target nodule is within a threshold proximity of the seed point. Based on a determination that the segmented candidate target nodule is within the threshold proximity of the seed point, the segmented candidate target nodule may be identified as an identified target nodule. A target nodule boundary corresponding to the identified target nodule may be displayed.

### CROSS-REFERENCED APPLICATIONS

This application claims priority to and benefit of U.S. Provisional Application No. 63/249,096, filed Sep. 28, 2021 and entitled “Systems and Methods for Target Nodule Identification,” which is incorporated by reference herein in its entirety.

### FIELD

Examples described herein are directed to systems and methods for graphically identifying a target nodule during the planning of an interventional procedure.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions clinicians may insert minimally invasive medical instruments (including surgical, diagnostic, therapeutic, or biopsy instruments) to reach a target tissue location. Some minimally invasive techniques use medical instruments that may be inserted into anatomic passageways and navigated toward a region of interest within the patient anatomy. Planning for such procedures may be conducted with reference to images of the patient anatomy that include the target tissue. Improved systems and methods may be used to identify the target tissue and plan an interventional procedure at the target tissue.

### SUMMARY

Consistent with some examples, a system may comprise one or more processors and memory having computer readable instructions stored thereon. The computer readable instructions, when executed by the one or more processors, may cause the system to receive image data including a segmented candidate target nodule, receive a seed point, and determine if the segmented candidate target nodule is within a threshold proximity of the seed point. Based on a determination that the segmented candidate target nodule is within the threshold proximity of the seed point, the segmented candidate target nodule may be identified as an identified target nodule. A target nodule boundary corresponding to the identified target nodule may be displayed.

In another example, a non-transitory machine-readable medium may comprise a plurality of machine-readable instructions which when executed by one or more processors associated with a planning workstation are adapted to cause the one or more processors to perform a method. The method may comprise receiving image data including a segmented candidate target nodule, receiving a seed point, and determining if the segmented candidate target nodule is within a threshold proximity of the seed point. Based on a determination that the segmented candidate target nodule is within the threshold proximity of the seed point, the segmented candidate target nodule may be identified as an identified target nodule. A target nodule boundary corresponding to the identified target nodule may be displayed.

It is to be understood that both the foregoing general description and the following detailed description are illustrative and explanatory in nature and are intended to provide an understanding of the present disclosure without limiting the scope of the present disclosure. Additional aspects, features, and advantages of the present disclosure will be apparent to one skilled in the art from the following detailed description.

### BRIEF DESCRIPTIONS OF THE DRAWINGS

FIG. 1 is a simplified diagram of a patient anatomy according to some examples.

FIG. 2 is a flowchart illustrating a method for planning an interventional procedure.

FIGS. 3-11 are simplified diagrams of a graphical user interface that may be used for planning an interventional procedure

FIG. 12 is a simplified diagram of a medical system according to some examples.

FIG. 13 is a simplified diagram of a side view of a patient coordinate space including a medical instrument mounted on an insertion assembly according to some examples.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

During the planning of a medical procedure using a steerable medical instrument, accurate target tissue identification may help to determine the most efficient and effective approach for accessing the interventional site. Pre-operative or intra-operative anatomical imaging data of the patient anatomy may be referenced to identify target tissue nodules, but fully automatic segmentation of target nodules from the imaging data may result in the identification of false target nodules or the omission of actual target nodules due to poor image quality, breathing motion, imaging artifacts, or other factors. However, purely manual identification of target nodules from the imaging data may be time consuming and inefficient. The systems and methods described herein may provide a hybrid identification approach that uses image segmentation to supplement the target tissue identification process. Because target tissue nodules may have irregular shapes that may not be approximated well by default symmetrical shapes such as spheres, circles, ellipses, and ellipsoids, the systems and methods described herein may allow a user to edit default shapes or the target tissue boundaries suggested by the image segmentation to better identify the irregular shape of the target nodule. Providing the planning system with accurate information about the size and shape of the target nodule may allow for procedure planning that fully considers one or more path options, multiple interventional points, and the proximity of vulnerable anatomic structures. Illustrative examples of a graphical user interface for planning a medical procedure, including but not limited to the lung biopsy procedures, are also provided below.

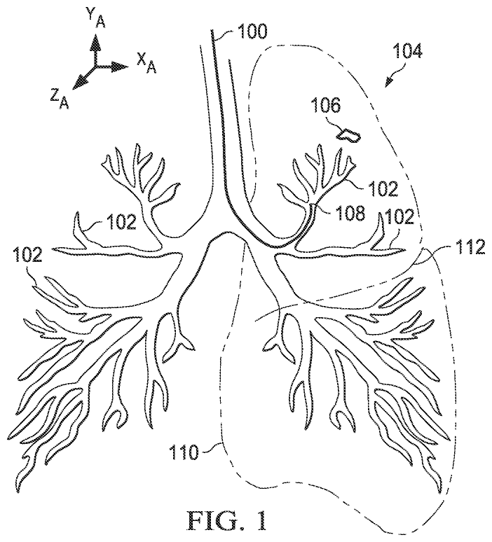

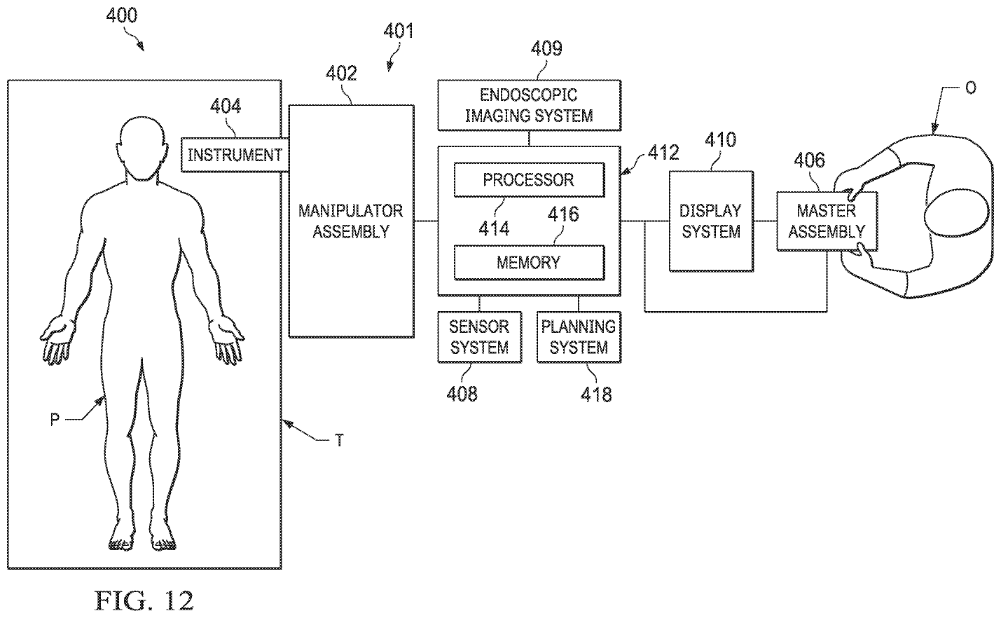

FIG. 1. illustrates an elongated medical instrument 100 extending within branched anatomic passageways 102 of an anatomical region 104 such as human lungs. The anatomical region 104 has an anatomical frame of reference (X_{A}, Y_{A}, Z_{A}). A distal end 108 of the medical instrument 100 may be advanced through the anatomic passageways 102 to perform a medical procedure, such as a biopsy procedure, at or near a target tissue, such as a target nodule 106. The anatomical region 104 may also include vulnerable surfaces or surfaces that are otherwise of interest when performing the medical procedure. For example, pulmonary pleurae 110 and pulmonary fissures 112 may be surfaces of interest because damaging these surfaces during the medical procedure may injure the patient. Before the medical procedure is performed, pre-operative planning steps may be conducted to plan the medical procedure. In some examples, a robot-assisted medical system (e.g. system 400, FIG. 12) may be used to plan and execute the medical procedure.

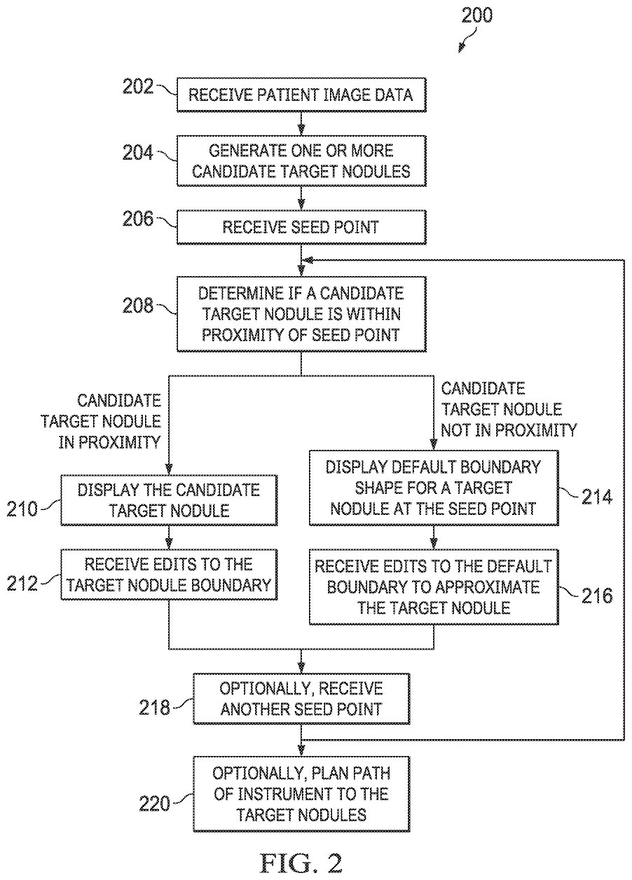

FIG. 2 illustrates a method 200 for identifying a target nodule during the planning of a medical procedure according to some examples. For example, planning a medical procedure may generally include planning trajectories between an initial tool location (e.g., an anatomical orifice such as a patient mouth) and one or more target nodules. One or more of the method steps may be performed on a procedure planning system (e.g. system 418, FIG. 12) that is incorporated into the same robotic-assisted medical system used to perform a biopsy or other medical procedure. Alternately or additionally, planning may be performed on a different system, such as a workstation dedicated to pre-operative planning. The plan for the medical procedure may be saved (e.g., as one or more digital files) and transferred to the robotic-assisted medical system used to perform the biopsy procedure. The saved plan may include the 3D model, identification of airways, target locations, trajectories to target locations, routes through the 3D model, and/or the like.

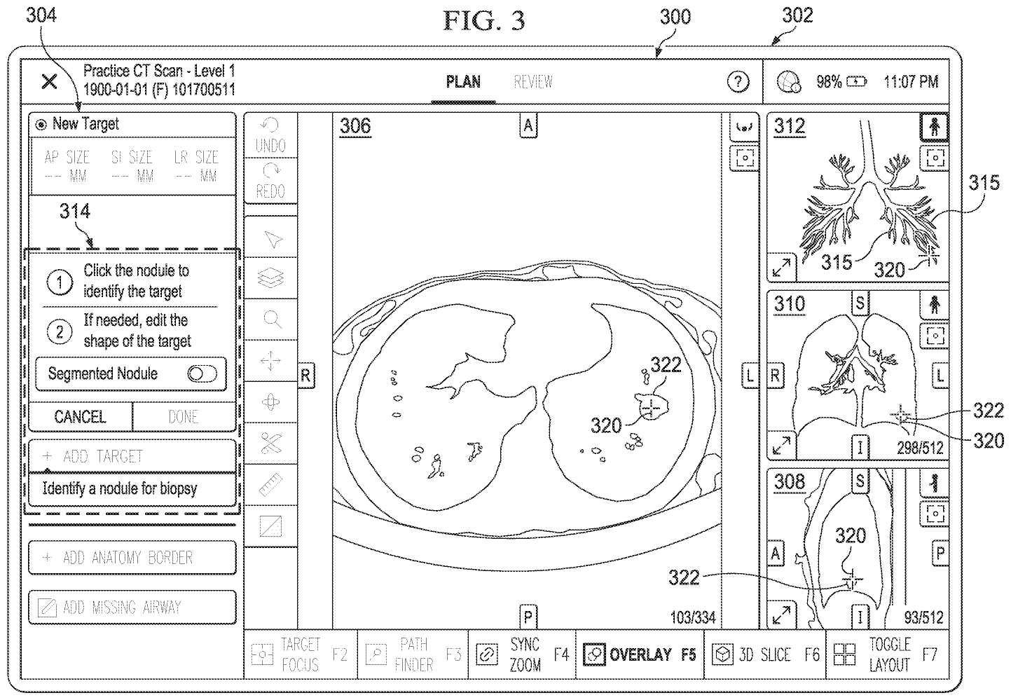

The method 200 may be illustrated as a set of operations or processes 202 through 220 and is described with continuing reference to FIGS. 3-11, which illustrates a graphical user interface 300 in a planning mode during the performance of method 200 according to some examples. At a process 202, patient image data may be received by a procedure planning system. The image data may include, for example, computed tomography (CT) image data and may be acquired pre-operatively or intra-operatively. For example, pre-operative image data may be received by conventional CT system, and intra-operative image data may be received from cone-beam CT system. In various alternative examples, image data may be generated using other imaging technologies such as magnetic resonance imaging (MRI), fluoroscopy, thermography, ultrasound, optical coherence tomography (OCT), thermal imaging, impedance imaging, laser imaging, nanotube X-ray imaging, and/or the like.

In some examples, the patient image data of an anatomical region may be displayed. For example, as illustrated in FIG. 3, the graphical user interface 300 may be displayed on a display system 302. The graphical user interface 300 may include a menu region 304 and one or more image panes such as an image pane 306, an image pane 308, an image pane 310, and an image pane 312 for presenting image data. In some examples, image pane 306 may display a cross-sectional view or “slice” of an anatomical region (e.g. anatomical region 104) selected from a series of cross-sectional views in an axial plane. In some examples, image pane 308 may display a cross-sectional view or “slice” of the anatomical region selected from a series of cross-sectional views in a saggital plane. In some examples, image pane 310 may display a cross-sectional view or “slice” of the anatomical region selected from a series of cross-sectional views in a coronal plane. In some examples, image pane 312 may display a three-dimensional model view of the anatomical region generated from segmented patient image data of the anatomical region. The image frames of reference of each of the views may be registered to each other such that a position in one view has a known, corresponding position in the other views.

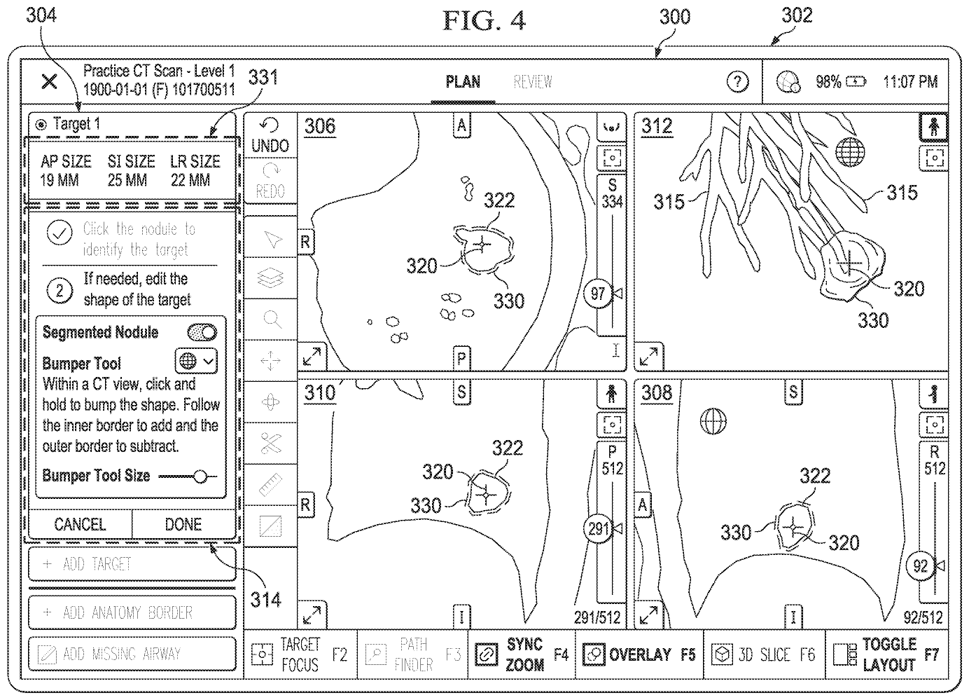

In some examples, additional panes may be displayed to present additional information or anatomic images. In some examples, fewer panes may be displayed. In some examples, the size or arrangement of the image panes 306-312 may be changed. For example, as shown in FIG. 4, the image panes 306-312 may be arranged in quadrants. Additionally or alternatively, the views of the anatomical region may be scaled or panned to alter the detail and/or portion of the anatomical region visible in the image pane. In other examples, the graphical user interface 300 may display any suitable number of views, in any suitable arrangement, and/or on any suitable number of screens. In some examples, the number of concurrently displayed views may be varied by opening and closing views, minimizing and maximizing views, moving views between a foreground and background of graphical user interface 300, switching between screens, and/or otherwise fully or partially obscuring views. Similarly, the arrangement of the views—including their size, shape, orientation, ordering (in a case of overlapping views), and/or the like—may vary and/or may be user-configurable.

Referring again to FIG. 2, at a process 204 one or more candidate target nodules may be generated by analyzing the image data. For example, the image data may be graphically segmented to delineate structures in the anatomical region that have characteristics associated with target nodules or other anatomic structures. The segmentation process may partition the image data into segments or elements (e.g., pixels or voxels) that share certain characteristics or computed properties such as color, density, intensity, and texture. In this example, various tissue structures including airways, candidate target nodules that may be associated with a pathology or other interest, pulmonary pleurae, pulmonary fissures, large bullae, and blood vessels may be segmented. The segmentation may be used to generate the three-dimensional model view of the anatomical region displayed in image pane 312. Various systems and methods for segmenting image data are described in U.S. Patent Application No. 63/171,996 (filed Apr. 7, 2021) (disclosing “User Interface for Connecting Model Structures and Associated Systems and Methods”); International Publication No. WO 2020/251958 A1 (filed Jun. 9, 2020) (disclosing “Detecting and Resenting Anatomical Features of an Anatomical Structure”); U.S. Pat. No. 10,373,719 B2 (filed Sep. 3, 2015) (disclosing “Systems and Methods for Pre-Operative Modeling”); U.S. Patent Application Pub. No. 2020/0043207 (filed Jul. 31, 2019) (disclosing “Systems and Methods for Generating Anatomical Tree Structures”); and International Publication No. WO 2020/186015 A1 (filed Mar. 12, 2020) (disclosing “Systems and Methods for Connecting Segmented Structures”), which are all incorporated by reference herein in their entireties.

In some examples, the display of one or more of the segmented structures or one or more categories of segmented structures may be suppressed. For example, in FIG. 3, the anatomic model in image pane 312 illustrates segmented anatomical passageways 315 but suppresses the display of segmented candidate target nodules and other pulmonary vasculature, pleurae, and fissures. Suppression of categories of segmented features may allow the displayed anatomical model to be adapted and simplified for a particular stage of the planning process. Suppression of segmented structures may also be useful when the image data may include irregularities due to image quality, breathing motion, imaging artifacts, or other factors that may result in segmentation of false features. The display of false features (i.e., false positives) may confuse or slow the identification and further investigation of target nodules. In other examples, some or all of the segmented features, including all of the candidate target nodules may be displayed.

Referring again to FIG. 2, at a process 206, a seed point associated with a target nodule (e.g., a target nodule 106) may be received based on a user input. For example and with reference to FIG. 3, a menu option 314 may be selected from the menu region 304. The menu option 314 may correspond to an ADD TARGET operation that allows a user to identify a target nodule for a procedure such as a biopsy. The user may scroll through cross-sectional views in the image panes 306, 308, 310 to visually identify the target nodule. For example, a target nodule 322 may be visible in one or more of the image panes 306, 308, 310. The user may then position an indicator 320, such as a crosshair marker or other cursor, at a seed point on a target nodule 322 visible in the image pane 306. In some examples, a user input for positioning the indicator 320 is received via a user input device. In some examples, the user input may be provided by the user via a user input device such as a mouse, a touchscreen, or a stylus. The user input device may have a frame of reference that is registered to the image frames of reference of the views shown in the image panes 308, 310, 312. The indicator 320 may also be displayed in the corresponding position in the other views shown in the image panes 308, 310, 312. In some examples, the seed point may be selected in any one of the image panes 306, 308, 310, 312, and the seed point will then be displayed in each of the other image panes at the corresponding position. The user's confirmation of the seed point at the position of the indicator 320 may be made by clicking, tapping, or otherwise providing confirmation via the user input device.

In some examples, guidance may be provided to the user to assist with determining where the indicator 320 should be positioned to locate the seed point. The guidance may, for example, take the form of previously analyzed or annotated radiology report or radiology images with identified target nodules. In other examples, the guidance may take the form of graphical cues, auditory tones, haptic forces or other hints that are provided when the indicator moves within a predetermined range of a candidate target nodule identified by the image segmentation. Additionally or alternatively, guidance may be provided in the form of region shading, region outlines, or other graphical cues that indicate the general region where candidate target nodules are located in the segmented image data.

Referring again to FIG. 2, at a process 208, the planning system may determine whether the seed point is on or within a proximity of a candidate target nodule. For example, the planning system may determine whether the seed point at the position of the indicator 320 is inside a candidate target nodule generated from the segmentation of the image data at process 204 or within a proximity of a candidate target nodule. The proximity may be a threshold proximity such as a predetermined proximity that is within a predetermined three-dimensional distance from the center or boundary of a candidate target nodule. Alternatively, the threshold proximity may be a distance that varies based upon the size of the candidate nodule, the distance from another candidate nodule, or other factors associated with the candidate nodule or the anatomic environment.

At a process 210, if a candidate target nodule is within the proximity of the seed point at process 208, the candidate target nodule may be displayed. The candidate target nodule may also be labeled, referenced, categorized, or otherwise recorded as an identified or selected target nodule within the planning system if the seed point is with the proximity of the candidate target nodule. Labels, user notes, characterizing information or other information associated with the selected target nodule may also be displayed. With reference to FIG. 4, a three-dimensional boundary 330 corresponding to a shape of a periphery of the segmented image data of the target nodule 322 may be displayed in the views in image panes 306, 308, 310, and/or 312. In the cross-sectional views of panes 306, 308, 310, the boundary 330 may have an irregular shape corresponding to the irregular outline of the boundary of the segmented target nodule 322. In the three-dimensional model view of image pane 312, the boundary 330 may be a three-dimensional representation of the segmented image data for the surface boundary of target nodule 322. The dimensions of the boundary 330, determined from the segmented image data, may be provided in a dimension display box 331. The dimensions may include, for example, a maximum anterior-posterior (AP) dimension, maximum superior-inferior (SI) dimension, and a maximum left-right (LR) dimension.

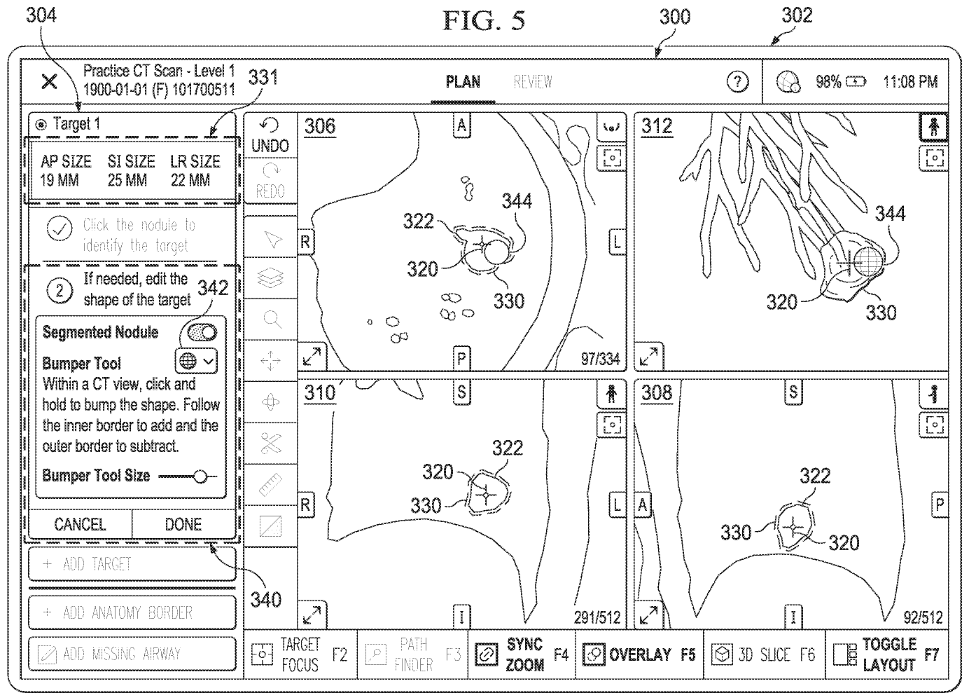

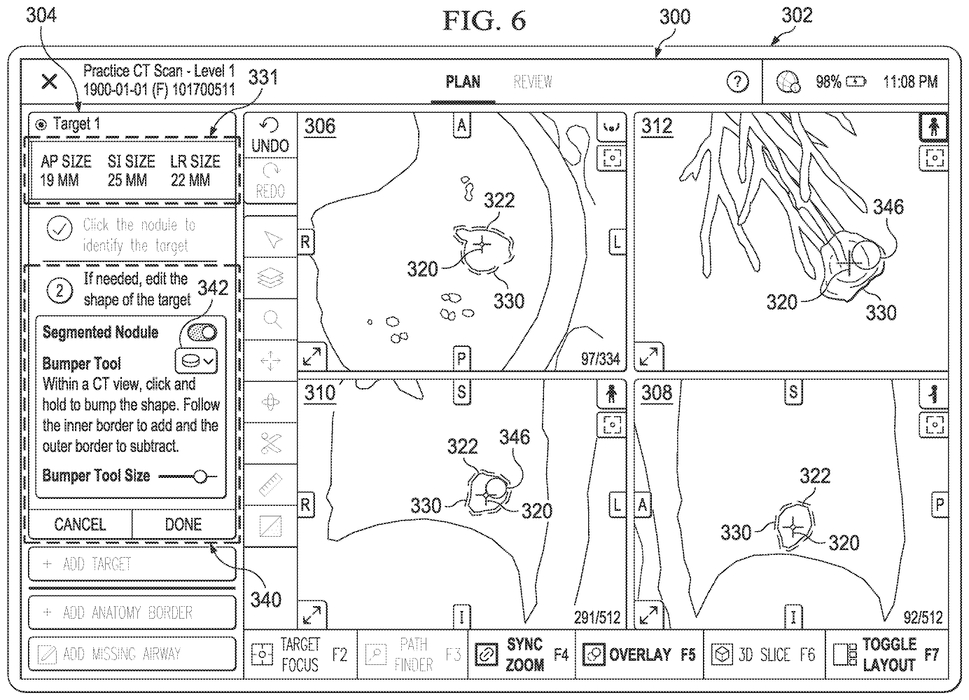

At a process 212, edits may be made to the boundary 330. For example, the user may observe that the segmented boundary does not correspond with the boundaries or nodule margins that the user observes in the cross-sectional views. The editing process allows the user to modify or revise the segmented boundary to better correspond to the boundaries and margins preferred by the user. With reference to FIG. 5, menu options 340 may be provided for editing the segmented shape 330 bounding the target nodule 322. A bumper tool selection menu 342 may allow the user to choose between different styles of tools for editing the segmented shape. One style of editing tool may be a multi-slice bumper tool 344 that may be displayed as a globe-shaped cursor in the three-dimensional view of image pane 312 and as a circle-shaped cursor in at least one of the two-dimensional cross-sectional views of image panes 306, 308, 310. As shown in FIG. 6, another style of editing tool may be a single-slice bumper tool 346 that may be displayed as a circle-shaped cursor in both the three-dimensional view of the image pane 312 and one of the two-dimensional cross-sectional views of panes 306, 308, 310. The size of the tools 344, 346 may be adjustable. If positioned within the segmented boundary 330, the editing tool 344, 346 may be moved into contact with the boundary 330 to extend or “bump out” the outline or boundary wall of boundary 330 at the location of the contact, thereby increasing the volume associated with the segmented target nodule 322. If positioned outside the segmented boundary 330, the editing tool 344, 346 may be moved into contact with the boundary 330 to reduce, retract, or “bump in” the outline or boundary wall of boundary 330 at the location of the contact, thereby decreasing the volume associated with the segmented target nodule 322. In other examples, the editing tools may take a different form such as an ellipse/ellipsoid shape, a pointer tool, a polygon shape, or any other shape that may be used to indicate an addition or reduction to the segmented shape. In other examples, other types of editing tools may be used to modify the boundary 330 to better align with the boundaries and nodule margins visualized by the user in the image data.

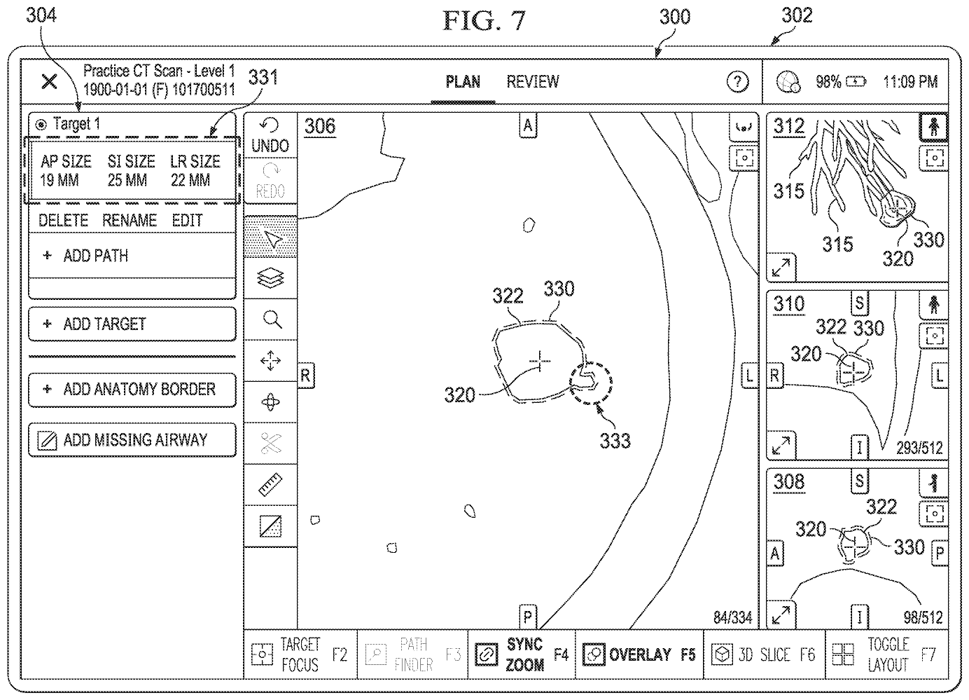

As shown in FIG. 7, during and/or after the editing process, the modified boundary 330 may be displayed. For example, the region 333, which was not originally identified as part of the target nodule 322, may be added to the segmented boundary 330 associated with the target nodule 322 based on the user's observation of the extent of the target nodule in the cross-sectional views. The user's subsequent boundary editing, as described above, adds the region 333 to be included within the modified boundary 330. The left-right (LR) dimension in the dimension display box 331 may also be updated to reflect the new size of the LR dimension after adding the region 333.

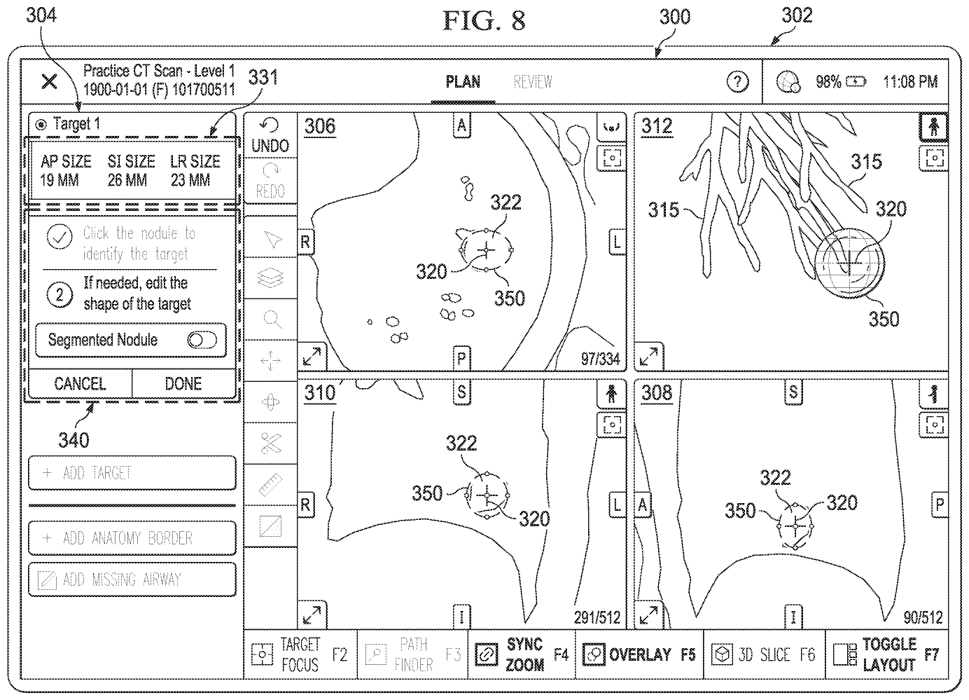

Referring again to FIG. 2, if a candidate target nodule is not within the proximity of the seed point at process 208, a candidate target nodule may not be displayed. Instead, at a process 214, a default boundary shape for a target nodule may be displayed at the seed point. With reference to FIG. 8, if a segmented candidate target nodule is not in the proximity of target nodule 322, a default boundary 350 may be displayed at the position of the seed point at indicator 320 to indicate the location of the target nodule 322. The boundary 350 may be displayed, for example, as an ellipsoid shape in the three-dimensional view of image pane 312 and may be displayed as a cross-section of the ellipsoid shape in the cross-sectional views in image panes 306, 308, 310. The boundary 350 may have a predetermined size and shape. In other examples, a sphere or other default shape may be used.

At a process 216, edits may be made to the size and/or shape of the boundary 350. In some examples the user may adjust the size of the boundary 350 by adjusting any of the three axial dimensions of the ellipsoid to approximate the dimensions of the target nodule 322. The adjustments may be based on the user's visualization of the target nodule 322 in the cross-sectional views of image panes 306, 308, 310. In some examples, the size of the ellipsoid shape may be adjusted to fully encompass the shape of the target nodule visible to the user in the cross-sectional views of image panes 306, 308, 310. In some examples, an editing tool such as the tool 344, 346 may be used to extend or reduce the boundary 350 into an irregular shape that more closely approximates the shape of the target nodule visible to the user in the cross-sectional views of image panes 306, 308, 310.

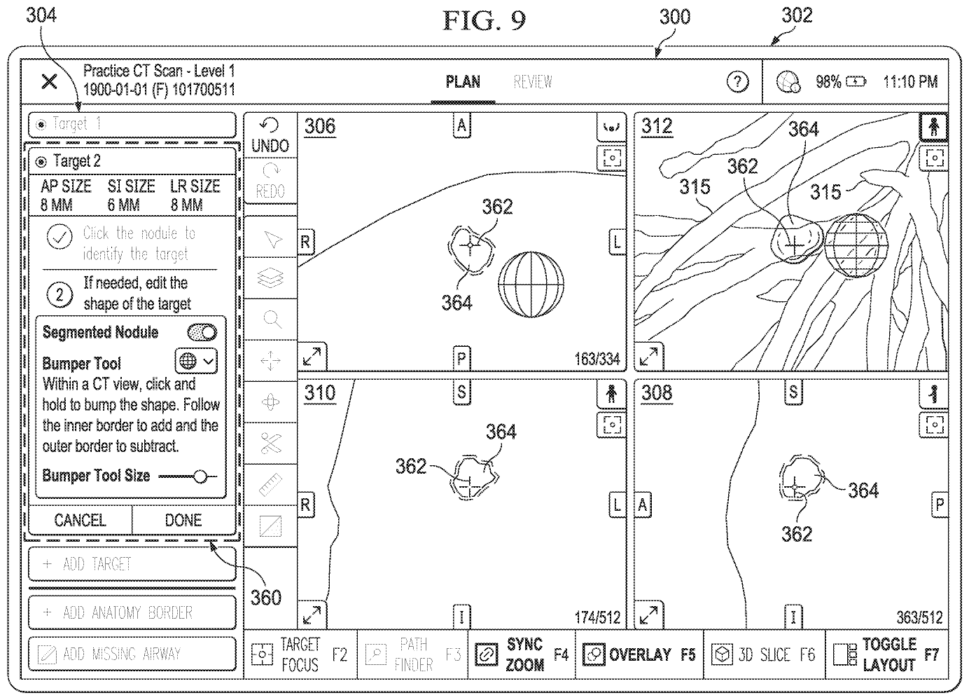

At an optional process 218, another seed point associated with a different target nodule may be received based on a user input. For example and with reference to FIG. 9, a menu option 360 may be selected from the menu region 304. The menu option 360 may correspond to an ADD TARGET 2 operation that allows a user to identify a second target nodule for a procedure such as a biopsy. As described for the identification of the first target nodule, the user may scroll through cross-sectional views in the image panes 306, 308, 310 to visually identify the second target nodule. The user may then position an indicator 362, such as a crosshair marker or other cursor, at a seed point on a target nodule 364 visible in the cross-sectional view in, for example, image pane 306. The processes 208-216 may then be repeated for the second seed point to refine the shape of the segmented and displayed boundary of the target nodule.

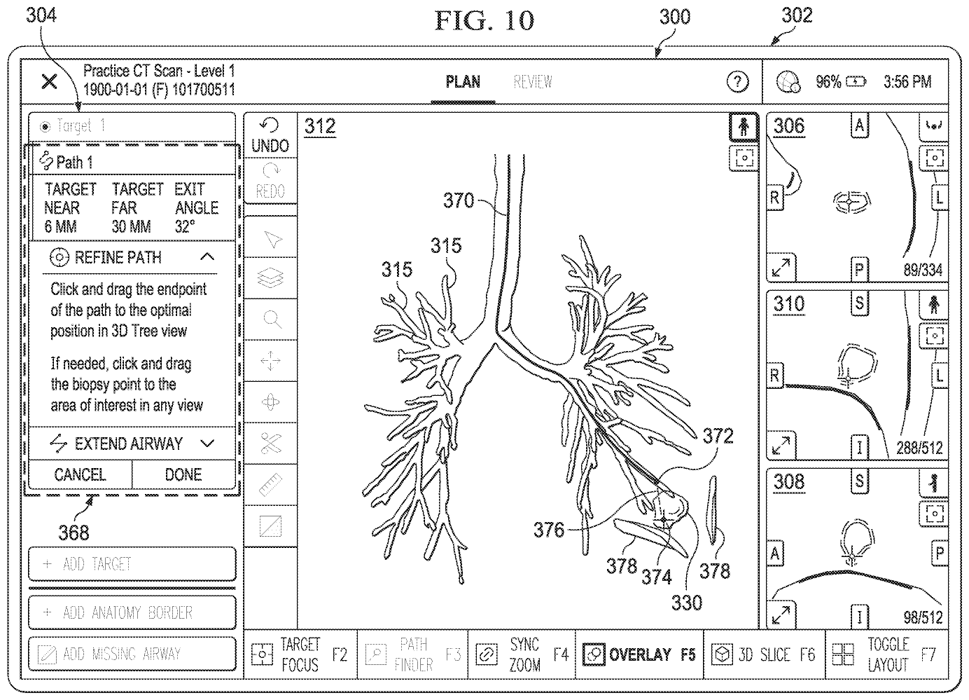

At an optional process 220, an instrument path to the target nodule may be planned. The instrument (e.g. instrument 100) may include, for example, a biopsy instrument for performing a biopsy procedure on the target nodule 322. The path may be generated manually by a user, automatically by the planning system, or by a combination of manual and automatic inputs. With reference to FIG. 10, a path planning menu 368 may be selected from the menu region 304 to plan a path 370 through the segmented airways 315 to the vicinity of the boundary 330 corresponding to the segmented image data of the target nodule 322. The planned path 370 may include a user indicated exit point 372 at which a flexible delivery instrument (e.g. a catheter) may be parked and from which a nodule engagement instrument (e.g., a biopsy needle) may exit the airways 315. The planned path 370 may also include a user indicated destination point 374. A trajectory line 376 may be displayed between the exit point 372 and the destination point 374. The destination point 374 may be a distal extent of the trajectory 376 of the nodule engagement instrument. In some examples, the destination point 374 may be, for example, the location at which the biopsy tissue is sampled. The destination point 374 may initially be indicated in the views of any of the panes 306-312 and then translated into the other views. In this example, anatomic structures that have been segmented and that may be vulnerable, such as pleurae 378 (e.g., the pleurae 110) may be displayed. The display of the category of vulnerable structures, which may include pleurae, vasculature, fissures, or the like) may be toggled between suppressed and displayed based upon user interest or based upon proximity to the exit or destination points.

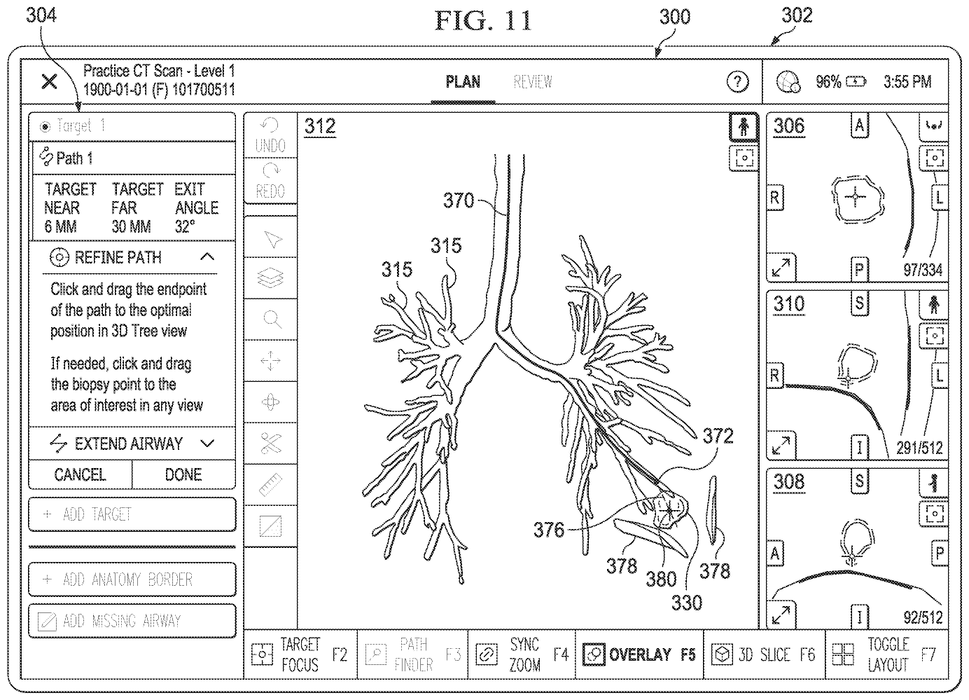

In an alternative example, the instrument path to the target nodule may be planned based on a user selected exit point and a destination point selected by the path planning system based on the configuration of the target nodule. For example, and with reference to FIG. 11, the planned path 370 may include the user indicated exit point 372 at which a flexible delivery instrument (e.g. a catheter) may be parked and from which a nodule engagement instrument (e.g., a biopsy needle) may exit the airways 315. In this example, the planned path 370 may also include a default destination point 380 that may be determined as a geometric center of the boundary 330 corresponding to the segmented image data of the target nodule 322. The destination point 380 may be a distal extent of the trajectory of the nodule engagement instrument. In some examples, the destination point 380 may be, for example, the location at which the biopsy tissue is sampled.

When a plurality of target nodules have been identified, the path planning system may identify paths, exit points, and destination points for each of the target nodules. The path planning may include planned efficiencies that minimize the airway traversal needed to reach multiple target nodules. In some examples, a target nodule may be biopsied in multiple locations and thus multiple paths, exit points, and destination points may be determined in planning a biopsy for a single target nodule. In some examples, a plurality of target nodules may be identified and displayed before the paths to each of the target nodules is planned. In other examples, the order of the processes 218, 220 may be swapped such that a path is planned after each target nodule is identified and displayed.

In some examples, the planning techniques of this disclosure may be used in an image-guided medical procedure performed with a teleoperated or robot-assisted medical system as described in further detail below. As shown in FIG. 12, a robot-assisted medical system 400 generally includes a manipulator assembly 402 for operating a medical instrument 404 in performing various procedures on a patient P positioned on a table T in a surgical environment 401. The medical instrument 404 may correspond to the instrument 100. The manipulator assembly 402 may be robot-assisted, non-robot-assisted, or a hybrid robot-assisted and non-robot-assisted assembly with select degrees of freedom of motion that may be motorized and/or robot-assisted and select degrees of freedom of motion that may be non-motorized and/or non-robot-assisted. A master assembly 406, which may be inside or outside of the surgical environment 401, generally includes one or more control devices for controlling manipulator assembly 402. Manipulator assembly 402 supports medical instrument 404 and may optionally include a plurality of actuators or motors that drive inputs on medical instrument 404 in response to commands from a control system 412. The actuators may optionally include drive systems that when coupled to medical instrument 404 may advance medical instrument 404 into a naturally or surgically created anatomic orifice. Other drive systems may move the distal end of medical instrument 404 in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to actuate an articulable end effector of medical instrument 404 for grasping tissue in the jaws of a biopsy device and/or the like.

Robot-assisted medical system 400 also includes a display system 410 (which may include display system 302) for displaying an image or representation of the surgical site and medical instrument 404 generated by a sensor system 408 and/or an endoscopic imaging system 409. Display system 410 and master assembly 406 may be oriented so an operator O can control medical instrument 404 and master assembly 406 with the perception of telepresence. Any of the previously described graphical user interfaces may be displayable on a display system 410 and/or a display system of an independent planning workstation.

In some examples, medical instrument 404 may include components for use in surgery, biopsy, ablation, illumination, irrigation, or suction. Optionally medical instrument 404, together with sensor system 408 may be used to gather (e.g., measure or survey) a set of data points corresponding to locations within anatomic passageways of a patient, such as patient P. In some examples, medical instrument 404 may include components of the imaging system 409, which may include an imaging scope assembly or imaging instrument that records a concurrent or real-time image of a surgical site and provides the image to the operator or operator O through the display system 410. The concurrent image may be, for example, a two or three-dimensional image captured by an imaging instrument positioned within the surgical site. In some examples, the imaging system components that may be integrally or removably coupled to medical instrument 404. However, in some examples, a separate endoscope, attached to a separate manipulator assembly may be used with medical instrument 404 to image the surgical site. The imaging system 409 may be implemented as hardware, firmware, software or a combination thereof which interact with or are otherwise executed by one or more computer processors, which may include the processors of the control system 412.

The sensor system 408 may include a position/location sensor system (e.g., an electromagnetic (EM) sensor system) and/or a shape sensor system for determining the position, orientation, speed, velocity, pose, and/or shape of the medical instrument 404.

Robot-assisted medical system 400 may also include control system 412. Control system 412 includes at least one memory 416 and at least one computer processor 414 for effecting control between medical instrument 404, master assembly 406, sensor system 408, endoscopic imaging system 409, and display system 410. Control system 412 also includes programmed instructions (e.g., a non-transitory machine-readable medium storing the instructions) to implement a plurality of operating modes of the robot-assisted system including a navigation planning mode, a navigation mode, and/or a procedure mode. Control system 412 also includes programmed instructions (e.g., a non-transitory machine-readable medium storing the instructions) to implement some or all of the methods described in accordance with aspects disclosed herein, including, for example, instructions for providing information to display system 410, instructions for determining a target location, instructions for determining an anatomical boundary, instructions for determining a trajectory zone, instructions for determining a zone boundary, and instructions for receiving user (e.g., operator O) inputs to a planning mode.

Robot-assisted medical system 400 may also include a procedure planning system 418. The procedure planning system 418 may include a processing system, a memory, a user input device, and/or a display system for planning an interventional procedure that may be performed by the medical system 400. In some examples, the planning system 418 incorporate other components of the medical system 400 including the control system 412, the master assembly 406, and/or the display system 410. Alternately or additionally, the procedure planning system 418 may be located at a workstation dedicated to pre-operative planning.

A plan for a medical procedure, such as a biopsy procedure, may be saved and used by the control system 412 to provide automated navigation or operator navigation assistance of a medical instrument to perform the biopsy procedure. Control system 412 may optionally further include a virtual visualization system to provide navigation assistance to operator O when controlling medical instrument 404 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like.

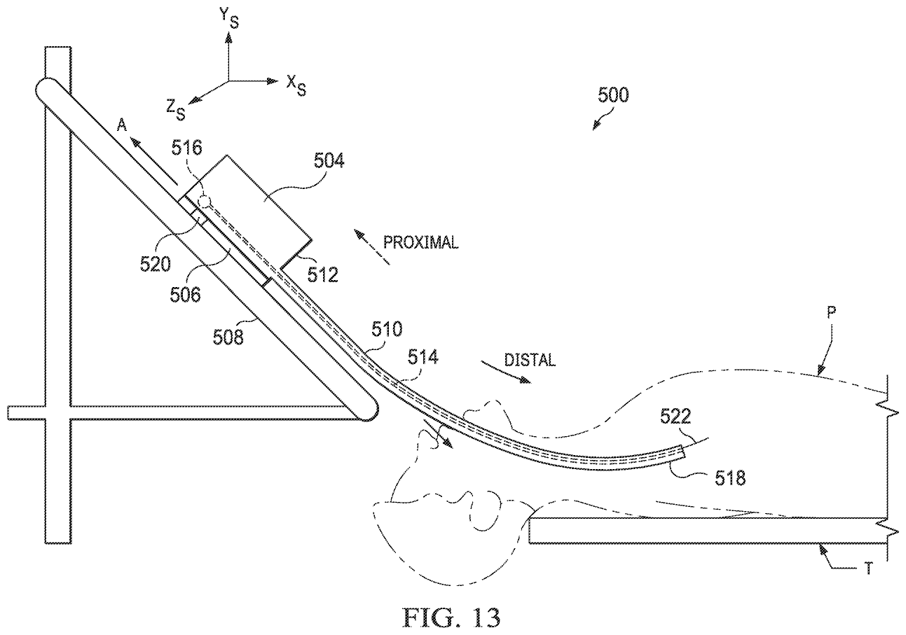

FIG. 13 illustrates a surgical environment 500 in which the patient P is positioned on the table T. Patient P may be stationary within the surgical environment in the sense that gross patient movement is limited by sedation, restraint, and/or other means. Cyclic anatomic motion including respiration and cardiac motion of patient P may continue unless the patient is asked to hold his or her breath to temporarily suspend respiratory motion. Within surgical environment 500, a medical instrument 504 (e.g., the instrument 100, 404), having the instrument frame of reference (X_{S}, Y_{S}, Z_{S}), is coupled to an instrument carriage 506. In this example, medical instrument 504 includes an elongate device 510, such as a flexible catheter, coupled to an instrument body 512. Instrument carriage 506 is mounted to an insertion stage 508 fixed within surgical environment 500. Alternatively, insertion stage 508 may be movable but have a known location (e.g., via a tracking sensor or other tracking device) within surgical environment 500. In these alternatives, the medical instrument frame of reference is fixed or otherwise known relative to the surgical frame of reference. Instrument carriage 506 may be a component of a robot-assisted manipulator assembly (e.g., manipulator assembly 402) that couples to medical instrument 504 to control insertion motion (i.e., motion along an axis A) and, optionally, motion of a distal end 518 of the elongate device 510 in multiple directions including yaw, pitch, and roll. Instrument carriage 506 or insertion stage 508 may include actuators, such as servomotors, (not shown) that control motion of instrument carriage 506 along insertion stage 508.

In this example, a sensor system (e.g., sensor system 408) includes a shape sensor 514. Shape sensor 514 may include an optical fiber extending within and aligned with elongate device 510. In one example, the optical fiber has a diameter of approximately 200 μm. In other examples, the dimensions may be larger or smaller. The optical fiber of shape sensor 514 forms a fiber optic bend sensor for determining the shape of the elongate device 510. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. patent application Ser. No. 11/180,389 (filed Jul. 13, 2005) (disclosing “Fiber optic position and shape sensing device and method relating thereto”); U.S. patent application Ser. No. 12/047,056 (filed on Jul. 16, 2004) (disclosing “Fiber-optic shape and relative position sensing”); and U.S. Pat. No. 6,389,187 (filed on Jun. 17, 1998) (disclosing “Optical Fibre Bend Sensor”), which are all incorporated by reference herein in their entireties. Sensors in some examples may employ other suitable strain sensing techniques, such as Rayleigh scattering, Raman scattering, Brillouin scattering, and Fluorescence scattering. In some examples, the shape of the catheter may be determined using other techniques.

As shown in FIG. 13, instrument body 512 is coupled and fixed relative to instrument carriage 506. In some examples, the optical fiber shape sensor 514 is fixed at a proximal point 516 on instrument body 512. In some examples, proximal point 516 of optical fiber shape sensor 514 may be movable along with instrument body 512 but the location of proximal point 516 may be known (e.g., via a tracking sensor or other tracking device) Shape sensor 514 measures a shape from proximal point 516 to another point such as distal end 518 of elongate device 510.

Elongate device 510 includes a channel (not shown) sized and shaped to receive a medical instrument 522. In some examples, medical instrument 522 may be used for procedures such as surgery, biopsy, ablation, illumination, irrigation, or suction. Medical instrument 522 can be deployed through elongate device 510 and used at a target location within the anatomy. Medical instrument 522 may include, for example, image capture probes, biopsy instruments, laser ablation fibers, and/or other surgical, diagnostic, or therapeutic tools. Medical instrument 522 may be advanced from the distal end 518 of the elongate device 510 to perform the procedure and then retracted back into the channel when the procedure is complete. Medical instrument 522 may be removed from proximal end of elongate device 510 or from another optional instrument port (not shown) along elongate device 510.

Elongate device 510 may also house cables, linkages, or other steering controls (not shown) to controllably bend distal end 518. In some examples, at least four cables are used to provide independent “up-down” steering to control a pitch of distal end 518 and “left-right” steering to control a yaw of distal end 518.

A position measuring device 520 may provide information about the position of instrument body 512 as it moves on insertion stage 508 along an insertion axis A. Position measuring device 520 may include resolvers, encoders, potentiometers, and/or other sensors that determine the rotation and/or orientation of the actuators controlling the motion of instrument carriage 506 and consequently the motion of instrument body 512. In some examples, insertion stage 508 is linear, while in other examples, the insertion stage 508 may be curved or have a combination of curved and linear sections.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions. Not all the illustrated processes may be performed in all examples of the disclosed methods. Additionally, one or more processes that are not expressly illustrated in may be included before, after, in between, or as part of the illustrated processes. In some examples, one or more of the processes may be performed by a control system or may be implemented, at least in part, in the form of executable code stored on non-transitory, tangible, machine-readable media that when run by one or more processors may cause the one or more processors to perform one or more of the processes.

Any alterations and further modifications to the described devices, instruments, methods, and any further application of the principles of the present disclosure are fully contemplated as would normally occur to one skilled in the art to which the disclosure relates. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for navigation and treatment of anatomic tissues, via natural or surgically created connected passageways, in any of a variety of anatomic systems, including the lung, colon, the intestines, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

One or more elements in examples of this disclosure may be implemented in software to execute on a processor of a computer system such as control processing system. When implemented in software, the elements of the examples of this disclosure may be code segments to perform various tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and/or magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11. Digital Enhanced Cordless Telecommunications (DECT), ultra-wideband (UWB). ZigBee, and Wireless Telemetry.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the invention are not described with reference to any particular programming language. It will be appreciated that a variety of programming languages may be used to implement the teachings of the invention as described herein.

This disclosure describes various instruments, portions of instruments, and anatomic structures in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

While certain illustrative examples of the invention have been described and shown in the accompanying drawings, it is to be understood that such examples are merely illustrative of and not restrictive on the broad invention, and that the examples of the invention not be limited to the specific constructions and arrangements shown and described, since various other modifications may occur to those ordinarily skilled in the art.

1. A system comprising:
one or more processors; and
memory having computer readable instructions stored thereon, the computer readable instructions, when executed by the one or more processors, cause the system to:
receive image data including a segmented candidate target nodule;
receive a seed point;
determine if the segmented candidate target nodule is within a threshold proximity of the seed point;
based on a determination that the segmented candidate target nodule is within the threshold proximity of the seed point, identify the segmented candidate target nodule as an identified target nodule; and
display a target nodule boundary corresponding to the identified target nodule.

2. The system of claim 1, wherein the computer readable instructions, when executed by the one or more processors, further cause the system to segment the image data to generate the segmented candidate target nodule.

3. The system of claim 1, wherein the image data includes pre-operative image data.

4. The system of claim 1, wherein the image data includes intra-operative image data.

5. The system of claim 1, wherein the image data includes pre-operative and intra-operative image data.

6. The system of claim 1, wherein receiving the seed point includes receiving three-dimensional coordinates corresponding to the seed point.

7. The system of claim 1, wherein the computer readable instructions, when executed by the one or more processors, further cause the system to provide guidance for positioning the seed point.

8. The system of claim 7, wherein the guidance includes a graphical representation of a region that includes the segmented candidate target nodule.

9. The system of claim 1, wherein the computer readable instructions, when executed by the one or more processors, further cause the system to receive user input to edit the displayed target nodule boundary, generate a revised target nodule boundary, and display the revised target nodule boundary.

10. The system of claim 9, wherein editing the displayed target nodule boundary includes extending a boundary wall if a cursor is inside the target nodule boundary.

11. The system of claim 9, wherein editing the displayed target nodule boundary includes retracting a boundary wall if a cursor is outside the target nodule boundary.

12. The system of claim 1, wherein the computer readable instructions, when executed by the one or more processors, further cause the system to display a default target indicator surrounding the seed point, wherein the displaying is based on a determination that the segmented candidate target nodule is not within the threshold proximity of the seed point.

13. The system of claim 12, wherein the default target indicator has a three-dimensional ellipsoid shape.

14. The system of claim 13, wherein the three-dimensional ellipsoid shape is adjustable.

15. The system of claim 1, wherein the computer readable instructions, when executed by the one or more processors, further cause the system to receive a target engagement location indicator for the identified target nodule.

16. The system of claim 15, wherein the computer readable instructions, when executed by the one or more processors, further cause the system to plan an instrument route to the target engagement location indicator.

17. A non-transitory machine-readable medium comprising a plurality of machine-readable instructions which when executed by one or more processors associated with a planning workstation are adapted to cause the one or more processors to perform a method comprising:
receiving image data including a segmented candidate target nodule;
receiving a seed point;
determining if the segmented candidate target nodule is within a threshold proximity of the seed point;
based on a determination that the segmented candidate target nodule is within the threshold proximity of the seed point, identifying the segmented candidate target nodule as an identified target nodule; and
displaying a target nodule boundary corresponding to the identified target nodule.

18. The non-transitory machine-readable medium of claim 17, wherein the method further comprises segmenting the image data to generate the segmented candidate target nodule.

19. The non-transitory machine-readable medium of claim 17, wherein the image data includes pre-operative image data.

20. The non-transitory machine-readable medium of claim 17, wherein the image data includes intra-operative image data.

21.-32. (canceled)
